(19) United States
(12) Patent Application Publication
(10) Pub. No.: US 2013/0259913 A1
(43) Pub. Date: Oct. 3, 2013

(54) PARTICLES AND PARTICLE GAS SATURATED SOLUTION PROCESSES FOR MAKING SAME

(71) Applicants: Holly Balasubramanian Rauckhorst, Ft. Thomas, KY (US); Qing Stella, Cincinnati, OH (US); Andreas Josef Dreher, Cincinnati, OH (US)

(72) Inventors: Holly Balasubramanian Rauckhorst, Ft. Thomas, KY (US); Qing Stella, Cincinnati, OH (US); Andreas Josef Dreher, Cincinnati, OH (US)

(21) Appl. No.: 13/433,336

(22) Filed: Mar. 29, 2012

(51) Int. Cl. A61K 8/02; C11D 3/60; C11D 17/00; A61K 8/86; A61K 8/64; A61K 8/72; A61K 8/35; A61K 8/33; A61K 8/36; A61K 8/92; A61K 8/891; A61Q 5/02; A61Q 19/00; A61Q 19/10; B32B 9/04; B05D 7/00; A61K 8/34; B82B 1/00; B82Y 99/00; B82Y 30/00

(52) U.S. Cl. 424/401; 514/772; 510/119; 510/137; 510/441; 510/506; 510/501; 510/475; 510/505; 510/488; 510/491; 510/466; 510/447; 428/402.24; 428/403; 427/212; 977/773; 977/926; 977/961; 977/902

(57) ABSTRACT

Particles containing at least two incompatible materials, such as a hydrophilic agent and a lipophilic agent, and particles from gas saturated solution (PGSS) processes for making such particles are provided.

### CROSS REFERENCE TO RELATED APPLICATION

This application claims the benefit of U.S. Provisional Application No. 60/474,000 filed Apr. 11, 2011.

### FIELD OF THE INVENTION

The present invention relates to particles and more particularly to particles comprising at least two materials, and even more particularly to particles comprising at least two incompatible materials and even still more particularly to particles comprising a hydrophilic agent and a lipophilic agent, and particles from gas saturated solution (PGSS) processes for making such particles.

### BACKGROUND OF THE INVENTION

Hydrophilic particles comprising hydrophilic agents, such as polyhydric alcohols, for example glycerin, are known in the art. Such hydrophilic particles have been utilized in aqueous systems, such as aqueous compositions and/or aqueous environments. The problem is that these hydrophilic particles tend to stay with the aqueous systems during use, which results in the hydrophilic particles oftentimes being washed down the drain before providing their full benefit.

Accordingly, there is a need for particles that comprise at least two incompatible materials, for example a hydrophilic agent and a lipophilic agent, that exhibit the benefits provided by at least one of the materials, but do not exhibit the negatives associated with the materials and a PGSS process for making such particles.

### SUMMARY OF THE INVENTION

The present invention fulfills the need by providing a particle comprising at least two incompatible materials and a PGSS process for making such particles.

In one example of the present invention, a particle comprising at least two incompatible materials, is provided.

In another example of the present invention, a particle comprising a hydrophilic agent and a lipophilic agent, is provided.

In still another example of the present invention, a particle comprising at least two incompatible materials, wherein the particle is produced by a PGSS process, is provided.

In another example of the present invention, a particle comprising at least two incompatible materials produced by a PGSS process, wherein the particle exhibits novel properties, is provided.

In yet another example of the present invention, a particle comprising a hydrophilic agent and a lipophilic agent, wherein the particle is produced by a PGSS process, is provided.

In another example of the present invention, a particle comprising a hydrophilic agent and a lipophilic agent, wherein the particle is produced by a PGSS process and wherein the particle exhibits novel properties, is provided.

In still yet another example of the present invention, a particle comprising glycerin and petrolatum, wherein the particle is produced by a PGSS process, is provided.

In even another example of the present invention, a process for producing a particle according to the present invention, wherein the process comprises depressurizing a solution comprising at least two incompatible materials and a highly compressible fluid dissolved in the solution such that a particle comprising the at least two incompatible materials is produced, is provided.

Accordingly, the present invention provides particles that comprise incompatible materials, for example a hydrophilic agent and a lipophilic agent, and such a particle produced by a PGSS process.

### BRIEF DESCRIPTION OF THE DRAWINGS

FIG. 1A is a schematic representation of an example of a particle according to the present invention;

FIG. 1B is a cross-sectional view of the particle of FIG. 1A;

FIG. 2A is a schematic representation of an example of a particle according to the present invention;

FIG. 2B is a cross-sectional view of the particle of FIG. 2A;

FIG. 3A is a schematic representation of an example of a particle according to the present invention;

FIG. 3B is a cross-sectional view of the particle of FIG. 3A;

FIG. 4A is a schematic representation of an example of a particle according to the present invention;

FIG. 4B is a cross-sectional view of the particle of FIG. 4A;

FIG. 5A is a schematic representation of an example of a particle according to the present invention;

FIG. 5B is a cross-sectional view of the particle of FIG. 5A; and

FIG. 6 is a schematic representation of a PGSS process for producing particles according to the present invention.

### DETAILED DESCRIPTION OF THE INVENTION

### Definitions

“Particle” as used herein means a composite, multi-component particulate or powder. In one example, the particle may be generally spherical in shape. In another example, the particle exhibits a Morphology Coefficient F of greater than 0.2 and/or greater than 0.4 and/or greater than 0.6 and/or greater than 0.8. In another example, the particle is a solid material produced from a PGSS process.

The particle may exhibit an average particle size of less than 500 μm and/or less than 250 μm and/or less than 100 μm and/or less than 50 μm and/or greater than 1 nm and/or greater than 100 nm and/or greater than 1 μm as measured according to the Particle Size Test Method described herein.

A plurality of particles of the present invention may exhibit an average particle size distribution from about 250 μm to 100 nm as measured according to the Particle Size Test Method described herein.

“Average particle size” as used herein for a material, such as a solid additive in accordance with the present invention, is determined according to the Particle Size Test Method described herein. The units for average particle size as used herein are μm.

“Incompatible” as used herein with reference to materials means that two or more materials phase separate when mixed together.

“Hydrophilic agent” as used herein means a material that exhibits a solubility of at least 5% and/or greater than 10% and/or greater than 30% and/or greater than 50% and/or greater than 75% to 100% by weight in distilled water. Solubility is defined as creation of a single phase from two or more materials at room temperature (23° C.±2.2°). Even if a material is does not meet the solubility criteria set forth above, the material may still be a hydrophilic agent if the material exhibits a contact angle of 80° or less and/or less than 75° and/or less than 70° and/or less than 60° and/or less than 50° and/or than about 25° and/or greater than about 30° and/or greater than about 35° and/or greater than about 40° as measured according to the Contact Angle Test Method described herein.

“Lipophilic agent” as used herein means a material that exhibits a solubility of less than 5% and/or less than 3% and/or less than 1% by weight in distilled water. Solubility is defined as creation of a single phase from two or more materials at room temperature (23° C.±2.2°). Even if a material is does not meet the solubility criteria set forth above, the material may still be a lipophilic agent if the material exhibits a contact angle of greater than 80° and/or greater than 90° and/or greater than 100° and/or greater than 110° and/or greater than 120° as measured according to the Contact Angle Test Method described herein.

“Non-ingestible” as used herein means that a material and/or particle is not suitable and/or intended for ingestion by a human and/or animal. For example, a non-ingestible particle is a particle that is not suitable and/or intended to be swallowed by a human and/or animal.

“Morphology Coefficient F” as used herein is a mathematical characterization of a particle of the present invention, for example a particle produced by a PGSS process. The Morphology Coefficient F of a particle is determined by the following equation:

$$Morphology Coefficient F = 6.9 \times {10}^{- 11} \frac{{( T , Kelvin )}^{4.247}}{{( p , bar )}^{0.403} \times {GTP}^{0.105}}$$

wherein p is the spraying pressure, T is the temperature pre-decompression, and GTP is the gas to particle product ratio.

### Particles

The particles of the present invention may comprise at least two incompatible materials. In one example, the particles of the present invention may comprise a hydrophilic material, such as glycerin, and a lipophilic material, such as petrolatum.

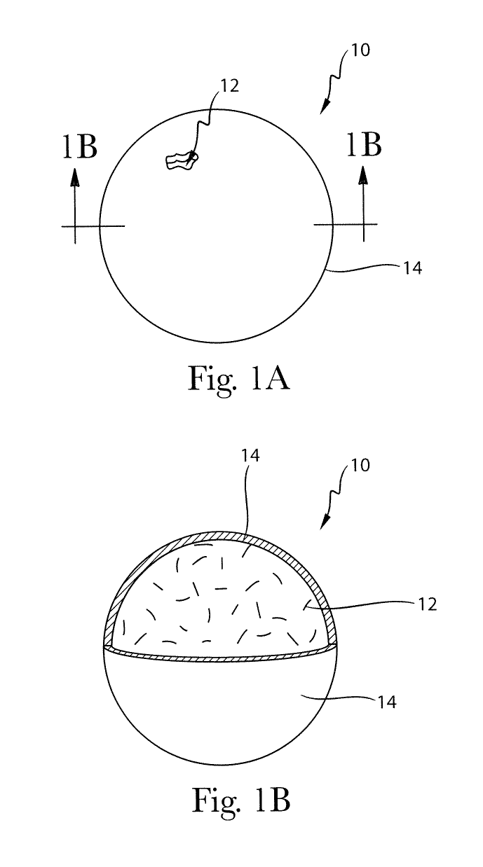

In one example as shown in FIGS. 1A and 1B, a particle 10 of the present invention may comprise a liquid core material 12, such as a hydrophilic material, for example glycerin, encapsulated within a solid shell material 14, such as a lipophilic material, for example petrolatum. The solid shell material 14 may be a non-porous shell such that the liquid core material 12 is not permitted to pass through the non-porous shell to the external environment until during use. Alternatively, the solid shell material 14 may be a porous shell such that the liquid core material 12 is capable of passing through the porous shell to the external environment. In one example, the solid shell material may at least partially encapsulate the liquid core material.

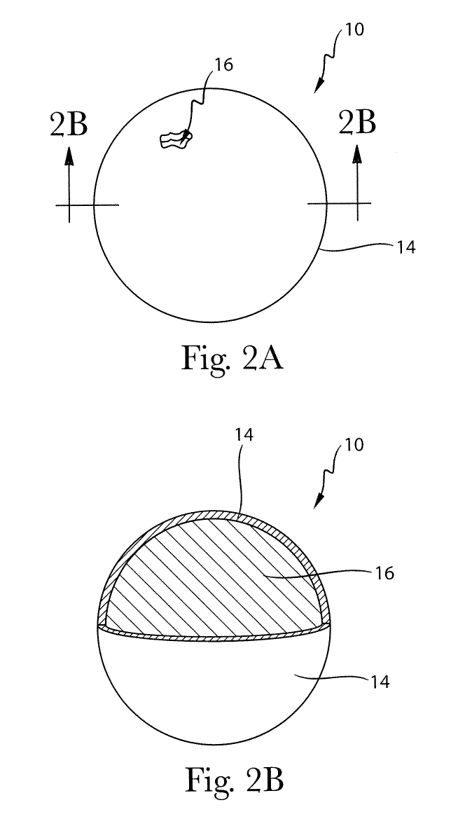

In another example as shown in FIGS. 2A and 2B, a particle 10 of the present invention may comprise a solid core material 16, such as a hydrophilic material, for example glycerin, encapsulated within a solid shell material 14, such as a lipophilic material, for example petrolatum. The solid shell material 14 may be a porous or non-porous shell. In one example, the solid shell material may at least partially encapsulate the solid core material.

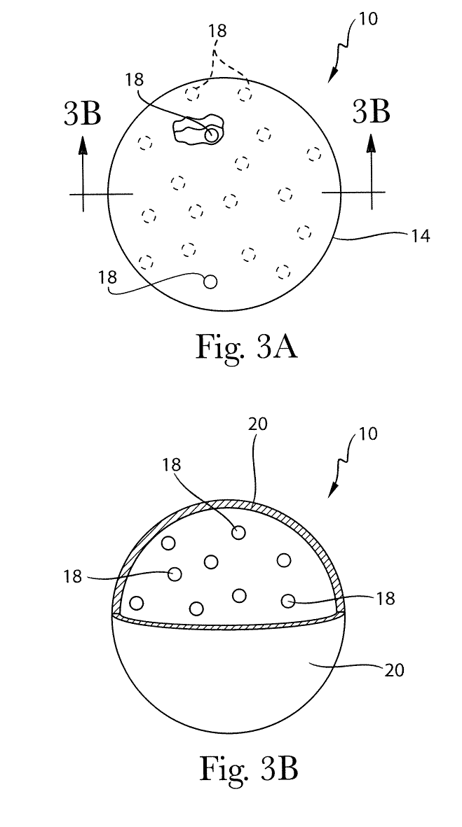

In even another example as shown in FIGS. 3A and 3B, a particle 10 of the present invention may comprise one or more liquid islands 18 of a material, such as a hydrophilic material, for example glycerin and a solid matrix material 20, such as a lipophilic material, for example petrolatum. In one example, the solid matrix material may at least partially encapsulate the one or more liquid islands.

In one example, the solid matrix material 20 of the present invention may be a gel, which may be a solid, jelly-like material. In one example, the solid matrix material 20 is a gel comprising a dispersion of solid particles within a liquid in which the solid particles constitute a discontinuous phase and the liquid constitutes a continuous phase.

In another example, the solid matrix material 20 of the present invention may be a colloid, where at least one material is microscopically dispersed evenly throughout another material.

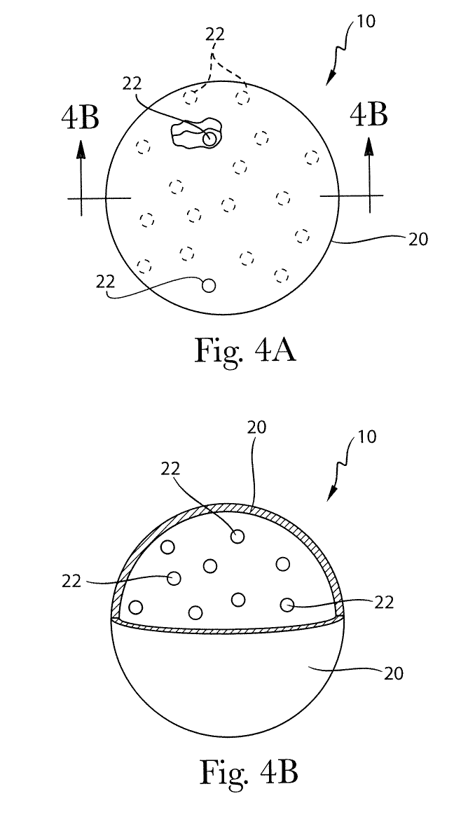

In even yet another example as shown in FIGS. 4A and 4B, a particle 10 of the present invention may comprise one or more solid islands 22 of a material, such as a hydrophilic material, for example glycerin and a solid matrix material 20, such as a lipophilic material, for example petrolatum. In one example, the solid matrix material may at least partially encapsulate the one or more solid islands.

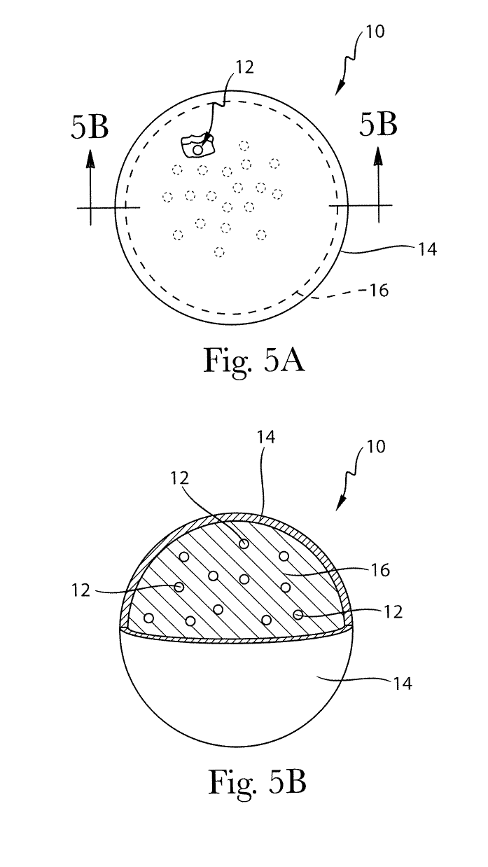

In still yet another example as shown in FIGS. 5A and 5B, a particle 10 of the present invention may comprise a mixture of liquid core material 12 and solid core material 16 dispersed within the liquid core material 12 and a solid shell material 14, which may be porous or non-porous. Alternatively, a solid matrix material may replace the solid shell material in this example. In one example, the solid shell material or solid matrix material may at least partially encapsulate the mixture of liquid core material and solid core material.

In addition to the configurations described above of the incompatible materials within the particle, the reverse configurations, such as the lipophilic material being a “core” material and the hydrophilic material being a “shell” material in the various examples shown in FIGS. 1 through 5, are also within the scope of the present invention.

The particle of the present invention may exhibit an average particle size of less than 500 μm and/or less than 250 μm and/or less than 100 μm and/or greater than 1 μm as measured according to the Particle Size Test Method described herein.

In one example, greater than 80% of a plurality of particles of the present invention exhibit a particle size of between 200 μm and 500 nm as measured according to the Particle Size Test Method described herein.

The particle exhibits a Morphology Coefficient F of greater than 0.2 and/or greater than 0.4 and/or greater than 0.6 and/or greater than 0.8.

In one example, the particle of the present invention comprises a weight ratio of hydrophilic material to lipophilic material of greater than 1:10 and/or greater than 1:5 and/or greater than 2:5 and/or greater than 1:2 and/or less than 10:1 and/or less than 5:1 and/or less than 5:2 and/or less than 2:1. In one example, the weight ratio of hydrophilic material to lipophilic material in a particle of the present invention is about 1:1.

In another example, the particle of the present invention may comprise greater than 5% and/or greater than 10% and/or greater than 20% and/or greater than 40% and/or less than 95% and/or less than 90% and/or less than 80% and/or less than 60% by weight of a hydrophilic material and less than 95% and/or less than 90% and/or less than 80% and/or less than 60% and/or greater than 5% and/or greater than 10% and/or greater than 20% and/or greater than 40% by weight of a lipophilic material.

In one example, a consumer product, for example a consumer product selected from the group consisting of: shampoos, body washes, laundry detergents, dishwashing detergents, anhydrous liquid products, bar soaps, paper products, cosmetics, lotions, skin treating products, and mixtures thereof, may comprise one or more particles of the present invention.

### Process for Making Particles

The particles of the present invention may be produced by a PGSS process. In one example of the present invention, a first material, for example a lipophilic material such as petrolatum and/or glycerol monooleate, may be mixed with a second material, for example a hydrophilic material such as glycerin and/or polyethylene glycol, to form a solution or an emulsion. From here forward we use solution or emulsion interchangeably. The first and second materials are under conditions such that they are present in the solution in their liquid states. Optionally, the solution can be pressurized to a pressure of at least 50 bars thus producing a pressurized solution. A highly compressible fluid may then be dissolved or partially dissolved in the solution thereby also pressurizing the system to 50 bar or higher. The pressurized solution is then rapidly depressurized as the solution is sprayed through a spray nozzle. During the depressurization and/or spraying, the highly compressible fluid is released from the solution and particles comprising the first material and second material are produced.

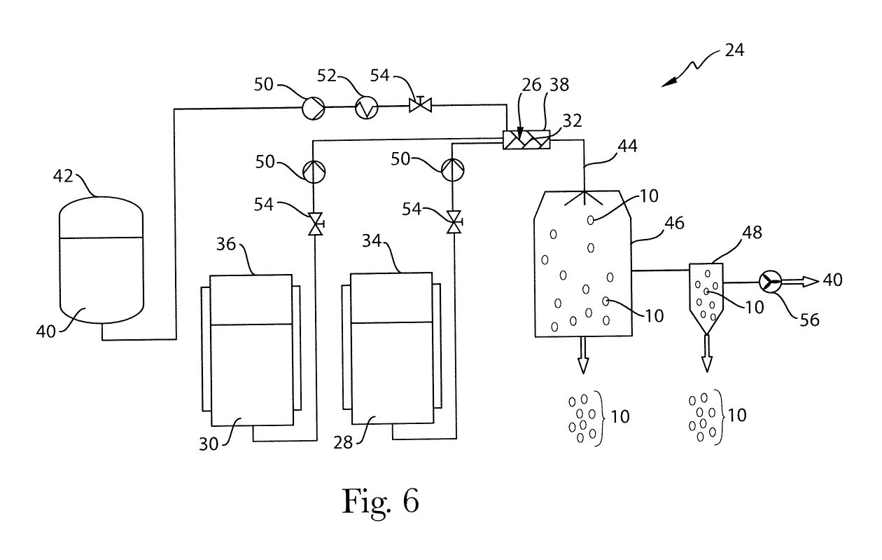

As shown in FIG. 6 a PGSS process 24 according to the present invention produces a particle and/or a plurality of particles 10. The PGSS process 24 comprises providing a pressurized solution 26 comprising at least a first material 28 and a second material 30. The first material 28 and second material 30 are mixed together to form a solution 32. The first material 28 may be sourced from a first storage vessel 34 and the second material 30 may be sourced from a second storage vessel 36. The solution 32 is pressurized and under conditions such that the first and second materials 28, 30 are in their liquid states. The first and second materials 28, 30 may be mixed together in a mixer 38, such as a static mixer, to form the solution 32.

A highly compressible fluid 40, which may be a liquid or a gas, is dissolved in the solution 32. The highly compressible fluid 40 may be sourced from a third storage vessel 42 and mixed with the first and second materials 28, 30 in the mixer 38.

After at least a portion of the highly compressible fluid 40 is dissolved into the solution 32, the solution 32 is then depressurized by spraying through one or more spray nozzles 44, such as within a spray tower 46. During the spraying operation, the solution 32 is depressurized and particles 10 are produced. The highly compressible fluid 40 is released from the solution 32 during the spraying operation. The highly compressible fluid 40 may have particles 10 entrained therein so it may be necessary to collect the particles 10 that are entrained in the highly compressible fluid 40. This collection may occur by passing the highly compressible fluid 40 through a cyclone filter 48 in order to separate the particles 10 from the highly compressible fluid 40 and increase the yield of the particles 10 produced by the PGSS process 24.

As shown in FIG. 6, the various components used in the PGSS process are fluidly connected to one another by any suitable piping, conduits, tubes, and the like. In one example, suitable pumps 50 may be used to help the flow of the materials within the process. In addition, a heat exchanger 52 may be utilized for one or more of the materials, for example the highly compressible gas 40. In still another example, stopcocks 54 may be used to manage the flow of the materials within the process. In still another example, a blower or fan 56 may be utilized within the process in order to help remove the highly compressible fluid 40 from the neat particles 10 produced in the process.

In addition to collecting the neat particles 10 as they are produced, the particles 10 may be collected in a slurry or suspension. In another example, the particles 10 may be mixed with a carrier in a Concentrated Powder Form (CPF) technology process. For example, a carrier, such as a waxy, powdery carrier is admixed into a stream of the particles 10 such that the particles contact and associate with the carrier to form a particle-charged carrier. The particle-charged carrier can then be collected. In one example, the average particle size of the carrier is less than 500 μm and/or less than 300 μm and/or less than 100 μm and/or less than 50 μm as measured by the Particle Size Test Method described herein. In other examples, the carrier may be a waxy or non-waxy solid at room temperature or a mineral, including silica or calcium carbonate.

In another example, the particles 10 and/or particle-charged carriers may be coated with a coating material to control the release of materials from the particles 10 and/or particle-charged carriers and/or influence the stability, such as shelf life, of the particles 10 and/or particle-charged carriers. The coating process may occur in a fluidized bed coater and/or a spray coating application process. In one example, coatings may be lipophilic or waxy materials such as paraffin. In another example, coatings may be aliphatic polymers such as polyethylene or polyethylene wax. Other non-limiting examples include poly(methyl methacrylate), or PMMA; poly(vinyl alcohol), or PVOH; poly(ethylene glycol), or PEG; and poly(ethylene oxide), or PEO. Non-limiting examples of suitable coating processes and/or materials are described in U.S. Pat. Nos. 6,221,826 and 7,338,928, both of which are incorporated herein by reference.

The PGSS process of the present invention thus produces particles from a solution, such as a liquid solution, producing a higher loading of hydrophilic in the lipophilic matrix in the particle structure, and a far lower highly compressible fluid content than was previously considered necessary for other known particle production processes using compressible fluids, such as RESS (rapid expansion from supercritical solutions). The cooling of the solution is so great, despite the unusually low highly compressible fluid content and high solution (incompatible materials) content, that the temperature falls below the solidification point of the solution to be treated downstream of the spray nozzle (decompression device). On decompression of a highly compressible fluid-containing solution in a suitable device, e.g. a commercially obtainable high-pressure spray nozzle, the highly compressible fluid is returned to the gaseous state and the solution (incompatible materials) to be treated precipitates as particles.

For the solidification point to be reached upon decompressing the solution it is necessary to comply with certain conditions. The melting point of the highly compressible fluid used should be at least 40 K and/or at least 80 K, and/or at least 100 K lower than the melting point of at least one and/or at least two and/or all the materials within the solution.

To assure that the cooling effect upon decompressing the solution is pronounced enough for particles to form, there has to be a certain minimum amount of highly compressible fluid dissolved in the solution. Depending on the solution to be treated and the type of highly compressible fluid used that minimum amount of highly compressible fluid dissolved in the solution may be from about 5% to about 90% and/or from about 8% to about 70% and/or from about 10% to about 50% by weight of the solution.

Further, the temperature of the highly compressible fluid-containing solution before decompression should be in the region of up to 50 K and/or up to 20 K and/or up to 10 K above or below the melting point of at least one and/or at least two and/or all of the materials within the solution under atmospheric pressure.

### Materials

The materials of the present invention may be any suitable materials known in the art. In one example, the materials comprise at least two incompatible materials. In another example, one of the materials is a hydrophilic agent. In another example, one of the materials is a lipophilic agent.

### Hydrophilic Material

The hydrophilic material of the present invention comprises a hydrophilic agent. Non-limiting examples of suitable hydrophilic agents include water, humectants, electrolytes, sugar amines, vitamins, such as Vitamin B families and Vitamin C families, natural extracts, protease inhibitors, α-hydroxyaldehydes and ketones, peptides, hexamidines, dehydroxy acetic acids, dihydroxy acetone, water-soluble polymers, water-swellable polymers, colorants, derivatives thereof, salts thereof, and mixtures thereof. In one example, the hydrophilic agent comprises a humectant. The humectant may comprise glycerin.

Non-limiting examples of suitable humectants include water, polyhydric alcohols, amino acids, pyrrolidone carboxylic acid and salt, hydroxyl acids, urea, urea derivatives and water soluble alkoxylated nonionic polymers, and mixtures thereof. Polyhydric alcohols useful herein include glycerin, sorbitol, propylene glycol, butylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof. Hydroxyl acids useful herein include lactic acid and glycolic acid, salicylic acid and their salts, and mixtures thereof. Water soluble alkoxylated nonionic polymers useful herein include polyethylene glycols and polypropylene glycols having a molecular weight of up to about 10000 such as those with CTFA names PEG-200, PEG-400, PEG-600, PEG-8000, and mixtures thereof.

Non-limiting examples of suitable electrolytes include sodium salts, potassium salts, calcium salts, and mixtures thereof.

Non-limiting examples of suitable sugar amines refer to amine derivatives of a six-carbon sugar, and are also known as amino sugars. Examples of sugar amines that are useful herein include glucosamine, N-acetyl glucosamine, mannosamine, N-acetyl mannosamine, galactosamine, and N-acetyl galactosamine, and mixtures thereof.

Non-limiting examples of suitable Vitamin B family components include vitamin B₃ compounds, such as niacinamide, nicotinic acid, nicotinyl alcohol, salts and/or derivatives, and mixtures thereof; and Vitamin B₅ compounds, such as a panthenoic acid derivative, including panthenol, dexpanthenol, ethyl panthenol, and mixtures thereof; and Vitamin B₆ compounds pyridoxine, esters of pyridoxine (e.g., pyridoxine tripalmitate), amines of pyridoxine (e.g., pyridoxamine), salts of pyridoxine (e.g., pyridoxine HCl) and derivatives thereof, including pyridoxamine, pyridoxal, pyridoxal phosphate, pyridoxic acid, and mixtures thereof.

Non-limiting examples of suitable Vitamin C family components include ascorbic acid and its salts, and ascorbic acid derivatives (e.g. magnesium ascorbyl phosphate, sodium ascorbyl phosphate, ascorbyl sorbate, ascorbyl glucoside) and mixtures thereof.

Non-limiting examples of suitable natural extracts include mulberry extract, placental extract, soy extract, green tea extract, chamomile extract, and mixtures thereof.

Non-limiting examples of suitable peptides, which refers to peptides containing ten or fewer amino acids and their derivatives, isomers and complexes with other species such as metal ions (e.g., copper, zinc, manganese, magnesium, and the like), include di-, tri-, tetra-, penta-, hexa-peptides, and derivatives and mixtures thereof.

Non-limiting examples of α-hydroxyaldehydes and ketones include dihydroxyacetone, glyceraldehydes, 2,3-dihydroxy-succindialdehyde, 2,3-dimethoxysuccindialdehyde, erythrulose, erythrose, 2-amino-3-hydroxy-succindialdehyde and 3-benzylamino-3-hydroxy-succindialdehye.

Non-limiting examples of suitable dehydroxyacetic acids and its salts, derivatives or tautomers include alkali metal salts, such as sodium and potassium; alkaline earth metal salts, such as calcium and magnesium; non-toxic heavy metal salts; ammonium salts; and trialkylammonium salts, such as trimethylammonium and triethylammonium, and mixtures thereof.

Non-limiting examples of suitable water-soluble or water-swellable polymer include homopolymers, copolymers or a blend of polymers or copolymers. The polymers can be natural, synthetic, or semi-synthetic. Polymers can be straight chain or cross-linked. Polymers, containing ionic and/or non-ionic groups, are contemplated. Ionic polymers include, but are not limited to, cationic, anionic, zwitterionic, and amphoteric polymers. The polymers can be synthesized from a variety of monomers containing unsaturated groups or by synthetic mechanisms that result in a variety of linking groups, including polyurethanes, polyesters, polyamides, and polyureas in the polymer backbone. Examples of useful commercially available synthetic polymers are listed below. The names described are according to the nomenclature developed by the Cosmetic, Toiletry, and Fragrance Association, Inc. (CTFA). In few cases, where the CTFA name is not available, the chemical name is written. Non-limiting examples include: vinylcaprolactam/PVP/dimethylamino-ethy lmethacry late copolymer (trade name: Gaffic, H2OLD, ISP Corp.), vinyl acetate/crotonic acid/vinyl propionate copolymer (trade name: Luviset, BASF), vinyl acetate/crotonates copolymer (trade name: Resyn, National Starch Corp.), vinyl acetate/butyl maleate/isobornyl acrylate copolymer (trade name: Advantage CPV, ISP), tyrene/vinyl pyrrolidone copolymer (trade name: Polectron, ISP); vinylpyrrolidone/vinyl acetate copolymers (ISP, BASF); polyvinylpyrrolidone/polyurethane interpolymer (Pecogel, Phoenix); octylacrylamide/acrylates/butylaminoethylmethacrylate copolymer (Amphomer, National Starch); quaternized vinylpyrrolidone/dimethylaminoethyl methacrylate copolymer (Polyquaternium-11, ISP), vinylpyrrolidone/vinyl acetate/vinyl propionate copolymer (Luviskol, BASF). In addition, other commercially available polymers listed in the Encyclopedia of Polymers and Thickeners, Cosmetic and Toiletries, page 95, Vol. 108, May 1993 can be included in this invention. Examples of natural and modified natural polymers are: copolymer of hydroxyethyl-cellulose and dimethyldiallyl ammonium chloride (Polyquaternium-4; National Starch), hydroxyethyl-cellulose (Natrosol; Aqualon), xanthan gum (Calgon), and other polymers listed in the Encyclopedia of Polymers and Thickeners, Cosmetic and Toiletries, page 95, Vol. 108, May 1993 can be included in this invention.

Silicone graft copolymers, hydrophobic graft copolymers and silicone block copolymer may also be useful as a water-soluble or water-swellable polymer.

The water-soluble or water-swellable polymers of the present invention may also include carboxylic acidlcarboxylate copolymers. The carboxylic acid/carboxylate copolymers herein can include cross-linked copolymers of carboxylic acid and alkyl carboxylate, and can have an amphophilic property. Commercially available carboxylic acid/carboxylate copolymers useful herein include: CTFA name Acrylates/C₁₀₋₃₀ Alkyl Acrylate Crosspolymer having tradenames Pemulene TR-1, Pemulene TR-2, Carbopol 1342, Carbopol 1382, and Carbopol ETD 2020, all available from B. F. Goodrich Company.

Non-limiting examples of suitable colorants include water soluble dyes. Water soluble dyes are dyes that are substantially soluble in aqueous solutions. Non-limiting examples of water soluble acid dyes include D& C Red 33, FD&C Yellow No. 5, D&C Green No. 5, D&C Yellow No. 8, and D&C Yellow No. 10. The colorants may also include an oxidizing agent (e.g. peroxides), and/or oxidative dye precursors (including developers and/or couplers when present).

### Structurants for Hydrophilic Material

The hydrophilic materials of the present invention may additionally contain a structurant. The inclusion of a structurant into the hydrophilic material increases the viscosity of the hydrophilic material. The combination of the hydrophilic material and structurant may form a mixture having a viscosity of at least about 3000 cst (centistokes) and/or at least 5000 cst at 25° C. as measured by a Brookfield Viscometer.

Non-limiting examples of suitable structurants for the hydrophilic material include surfactants, polymers (such as polysaccharides and alkoxylated polymers), fluid absorbent particles, inorganic particulate thickeners, and water-soluble or water-swellable polymers. In one example, the ratio of structurant to hydrophilic material is from about 1:1000 to about 100:1 and/or from about 1:200 to about 80:1 and/or from about 1:100 to about 50:1 and/or from about 1:20 to about 20:1.

1. Surfactants

Non-limiting examples of suitable surfactants that can be used as structurants include anionic surfactants, nonionic surfactants, cationic surfactants, amphoteric surfactants and mixtures thereof.

Non-limiting example of suitable anionic surfactants include sulfonates such as alkane sulfonates (e.g., branched sodium x-alkane sulfonate where x≠1), paraffin sulfonates, alkylbenzene sulfonates, α-olefin sulfonates, sulfosuccinates and sulfosuccinate esters (e.g., dioctylsodium and disodium laureth sulfosuccinate), oisethionates, acylisethionates (e.g., sodium 2-lauroyloxyethane sulfonate), and sulfalkylamides of fatty acids, particularly N-acylmethyltaurides; sulfates such as alkyl sulfates, ethoxylated alkyl sulfates, sulfated monoglycerides, sulfated monoglycerides, sulfated alkanolamides, and sulfated oils and fats; carboxylates such as alkyl carboxylate having a carbon chain length above C₁₂, acylsarcosinates, sarcosinates (e.g., sodium lauryl sarcosinate), ethoxylated carboxylic acid sodium salts, carboxylic acids and salts (e.g., potassium oleate and potassium laurate), ether carboxylic acids; ethoxylated carboxylic acids and salts (e.g., sodium carboxymethyl alkyl ethoxylate; phosphoric acid esters and salts (e.g., lecithin); acylglutamates (e.g., disodium n-lauroyl glutamate) and mixtures thereof. It should be noted that the safest alkyl sulfates for use generally have hydrocarbon chain lengths above C₁₂.

Non-limiting examples of suitable nonionic surfactants include polyoxyethylenes such as ethoxylated fatty alcohols, ethoxylated alcohols (e.g., octaoxyethelene glycol mono hexadecyl ether, C₁₆E₈ and C₁₂E₈), ethoxylated fatty acids, ethoxylated fatty amines, ethoxylated fatty amides, ethoxylated alkanolamides, ethoxylated alkyl phenol, and ethoxylated sterols; triesters of phosphoric acid (e.g., sodium dioleylphosphate); alkyl amido diethylamines; alkylamido propylbetaines (e.g., cocoamido propylbetaine); amine oxide derivatives such alkyl dimethylamine oxides, alkyl dihydroxyethylamine oxides, alkyl amidodimethylamine oxidesand alkyl amidodihydroxyethylamine oxides; polyhydroxy derivatives such as polyhydric alcohol esters and ethers (e.g., sucrose monooleate, cetostearyl glucoside, β octyl glucofuranoside, esters, alkyl glucosides having a carbon chain length of from C₁₀ to C₁₆), mono, di- and polyglycerol ethers and polyglycerol esters (e.g., tetraglycerol monolaurate and monoglycerides, triglycerol monooleate (such as TS-T122 supplied by Grinsted), diglycerol monooleate (such as TST-T101 supplied by Grinsted), ethoxylated glycerides; monoglycerides such as monoolein, monolaurin and monlinolein; diglyceride fatty acids such as diglycerol monoisostearate (e.g., Cosmol 41 fractionated supplied by Nisshin oil Mills, Ltd.) and mixtures thereof.

Non-limiting examples of suitable cationic surfactants include aliphatic-aromatic quaternary ammonium halides; quaternary ammonium alkyl amido derivatives; alkyl amidopropyldimethylammonium lactate; alkylamidopropyldihydroxyethylammo-nium lactate; alkyl amidopropyl morpholinium lactate; quaternary ammonium lanolin salts; alkyl pyridinium halides; alkyl isoquinolinium halides; alkyl isoquinolinium halides; quaternary ammonium imidazolinium halides; bisquaternary ammonium derivatives; alkylbenzyl dimethylammonium salts such as stearalkylammonium chloride; alkylbetaines such as dodecyldimethylammonium acetate and oleylbetaine; alkylethylmorpholinium ethosulfaates; tetra alkyl ammonium salts such as dimethyl distearyl quaternary ammonium chloride and bis isostearamideopropyl hydroxypropyl diammonium chloride (Schercoquat 2IAP from Scher Chemicals); heterocyclic ammonium salts; bis(triacetylammoniumacetyl)diamines and mixtures thereof.

Non-limiting example of suitable amphoteric surfactants include alkyl betaines; alkanolamides such as monoalkanolamides and dialkanolamides; alkyl amido propylbetaines; alkyl amidopropylhydroxysultaines; acylmonocarboxy hydroxyethyl glycinates; acyldicarboxy hydroxyethyl glycinates; alkyl aminopropionates such as sodium laurimino dipropionate; alkyl iminodipropionates; amine oxides; acyl ethylenediamine betaines; N-alkylamino acids such as sodium N-alkylamino acetate; N-lauroylglutamic acid cholesterol esters; alkyl imidazolines and mixtures thereof.

Silicone copolyols, for example DC-190, DC-193, DC5329, Q4-3667 from Dow Corning; and aminosilicones, for example Silwet L-7622 and Silwet L-77 from Union Carbide can also be used as structurants in the present invention.

2. Polymers

In addition to the surfactants, certain polymers such as alkoxylated polymers and polysaccharides may be used as structurants in the present invention. The polymers may have a molecular weight of from about 500 to about 1,000,000 and/or from about 750 to about 500,000 and/or from about 1,000 to about 60,000.

Non-limiting example of suitable polysaccharides include polyglucose materials, gums, hydrocolloids, cellulose and cellulose-derivative polymers. Many of these and other suitable polysaccharides are described in Industrial Gums—Polysaccharides and Their Derivatives, Roy L. Whistler, Academic Press (New York), 1959 and also in P. Weigel et al., “Liquid Crystalline States in Solutions of Cellulose and Cellulose Derivatives,” Acta Polymerica Vol. 35 No. 1, 1984, pp. 83-88. In addition, other suitable polysaccharides include nonionic, anionic and cationic polysaccharides.

Non-limiting examples of nonionic polysaccharides include hydroxypropyl cellulose polymers, examples of which are available from Hercules, Inc. under the trade name KLUCEL and xantham gum available from Kelco.

Non-limiting examples of anionic polysaccharides include sodium alginates (available from Kelco) and sodium carboxymethylcellulose polymer available from Hercules, Inc.

Non-limiting examples of suitable cationic polysaccharides include chitosan and/or chitin available from Protan, Inc, and also depolymerized guar, such as T4406 from Hi Tek Polymers.

Non-limiting examples of suitable alkoxylated polymers include the Poloxamer Series of EO-PO condensates (A-B-A type block copolymers of polyoxyethylene and polyoxypropylene). Suitable examples of polyoxyethylene-polyoxypropylene block copolymers include Poloxamers 403, 402, and 401 available under the trade names PLURONIC P123, PLURONIC L-122, and PLURONIC L-121 from BASF and Hodag Nonionic 1123-P and Hodan Nonionc 1122-L from Calgene and SYNPERONIC PE/L121 from ICI.

3. Fluid Absorbent Particles

Suitable fluid absorbent particles include particles that have an average particle size of from about 0.001 microns to about 2000 microns and/or from about 0.01 microns to about 200 microns and/or from about 0.1 microns to about 100 microns. Non-limiting examples of suitable fluid absorbent particles include silicas (or silicon dioxides), silicates, carbonates, various organic copolymers, and combinations thereof. The silicates are most typically those formed by reaction of a carbonate or silicate with an alkali metal, alkaline earth metal, or transition metal, specific non-limiting examples of which include calcium silicate, amorphous silicas (e.g., precipitated, fumed, and colloidal), calcium carbonate (e.g., chalk), magnesium carbonate, zinc carbonate, and combinations thereof. Non-limiting examples of some suitable silicates and carbonates for use herein are described in Van Nostrand Reinhold's Encyclopedia of Chemistry, 4^{th} edition, pages 155, 169, 556, and 849 (1984). Absorbent powders are also described in U.S. Pat. No. 6,004,584.

Other fluid-absorbent particles suitable for use herein include kaolin, (hydrated aluminum silicates), mica, talc (hydrated magnesium silicates), starch or modified starch, microcrystalline cellulose (e.g., Avicel from FMC Corporation), or other functionally similar fluid-absorbent polymer, any other silica-containing or non-silica-containing powder.

Other fluid-absorbent particles suitable for the use herein include super-absorbent polymers. By definition, a superabsorbent polymer must absorb a minimum of 20 times its own weight in water. Moreover, the polymer must retain its original identity and have sufficient physical integrity to resist flow and fusion with neighboring particles, and to swell to equilibrium volume and not dissolve. Non-limiting examples include Water Lock® superabsorbent polymers (e.g. Starch graft poly (2-propenamide-co-2-propenoic acid) sodium or potassium salt, 2-propenamide-co-2-propenoic acid copolymer, sodium salt) manufactured by Grain Processing Corporation.

4. Inorganic Particulate Thickeners

Non-limiting examples of suitable inorganic particulate thickeners include silica and clay (e.g. Benton clays from Rhox) with particle sizes less than 1 micron.

5. Water-Soluble or Water-Swellable Polymers

Non-limiting examples of suitable water-soluble or water swellable polymers include those described herein above.

### Lipophilic Material

The lipophilic material comprises a lipophilic agent. Non-limiting examples of suitable lipophilic agents include ester lipids, hydrocarbon lipids, silicone lipids, fatty alcohols, fatty acids, and mixtures thereof.

Non-limiting examples of suitable ester lipids include lipids that have at least one ester group in the molecule. One type of common ester lipids useful in the present invention are the fatty acid mono and polyesters such as cetyl octanoate, octyl isonanoanate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate, sucrose esters (such as sucrose esters derived from fatty acids) and polyesters, sorbitol ester, and the like.

In one example, the lipophilic material comprises glyceryl monooleate.

In another example, the lipophilic material comprises paraffin and/or a microcrystalline wax.

Another type of ester lipid suitable for the present invention includes triglycerides and modified triglycerides, and mixtures thereof. These include vegetable oils such as jojoba, soybean, canola, sunflower, safflower, rice bran, avocado, almond, olive, sesame, persic, castor, coconut, and mink oils. Synthetic triglycerides can also be employed. Modified triglycerides include materials such as ethoxylated and maleated triglyceride derivatives. Proprietary ester blends such as those sold by Finetex as Finsolv are also suitable, as is ethylhexanoic acid glyceride.

A third type of ester lipid is liquid polyester formed from the reaction of a dicarboxylic acid and a diol. Examples of polyesters suitable for the present invention are the polyesters marketed by ExxonMobil under the trade name PURESYN ESTER®.

Non-limiting examples of suitable hydrocarbon lipids, which may be liquid or semi-solid hydrocarbons, include linear and branched oils such as liquid paraffin, squalene, squalane, mineral oil, low viscosity synthetic hydrocarbons such as polyalphaolefin sold by ExxonMobil under the trade name of PURESYN PAO and polybutene under the trade name PANALANE or INDOPOL, and mixtures thereof. Light (low viscosity), highly branched hydrocarbon oils are also suitable.

Petrolatum is an example of a hydrocarbon lipid that is suitable for the present invention. Its semi-solid nature can be controlled both in production and by the formulator through blending with other oils or fractionating to remove one or more of the hydrocarbon components from the blend, such as eliminating lower chains (for example C₂₀-C₃₆). Petrolatum is often described as a “complexed mixture of cyclic, branched, and linear hydrogenated hydrocarbon oils and waxes commonly referred to as mineral oils, paraffin and microcrystalline waxes”. In one example, the petrolatum is void or significantly void of all lower chains (for example C₂₀-C₃₆) white oils & cyclic paraffins, which have been replaced with a higher viscosity mineral oil having longer chains (for example C₄₀-C₅₀), for example Hydrobrite 1000, which is commercially available from R.E. Carroll, Inc., Trenton, N.J. Additionally, the level of microcrystalline wax (having chain lengths of from about C₃₀-C₇₅) can be increased to stabilize the oils at room temperature (about 23° C.) and to provide the needed lipid structure at elevated temperatures. This petrolatum may exhibit a melting point of from about 135° F. to about 155° F. and a viscosity at 210° F. of 80 centipoise or greater as measured by a Brookfield Viscometer.

Another example of a suitable petrolatum is known in the art as Super White Petrolatum. It exhibits a melting point of from about 130° F. to about 140° F. and a viscosity at 210° F. of less than 80 centipoise as measured by a Brookfield Viscometer.

In another example, a polymer-modified petrolatum, such as Versagel P200 commercially available from Penreco, Houston, Tex., is suitable for use in the present invention. This petrolatum contains a polymer thickening agent, which may serve to increase the viscosity of the petrolatum.

Non-limiting examples of suitable silicone lipids include linear and cyclic polydimethyl siloxane, organo functional silicones (alkyl and alkyl aryl), and amino silicones, and mixtures thereof.

Non-limiting example of suitable fatty alcohols include liquid fatty alcohols having from about 10 to about 30 carbon atoms. These liquid fatty alcohols may be straight or branched chain alcohols and may be saturated or unsaturated alcohols. Liquid fatty alcohols are those fatty alcohols which are liquid at about 25° C. Non-limiting examples of these compounds include oleyl alcohol, palmitoleic alcohol, isostearyl alcohol, isocetyl alcohol, and mixtures thereof.

Non-limiting examples of suitable fatty acids include liquid fatty acids having from about 10 to about 30 carbon atoms. These fatty acids can be straight or branched chain acids and can be saturated or unsaturated. Suitable fatty acids include, for example, oleic acid, linoleic acid, isostearic acid, linolenic acid, ethyl linolenic acid, arachidonic acid, ricinolic acid, and mixtures thereof.

### Stabilizing/Emulsifying Agents

In addition to hydrophilic and lipophilic materials described above, one or more stabilizing and/or emulsifying agents may be mixed with the hydrophilic and/or lipophilic materials to aid in the formation of the solution comprising the hydrophilic and lipophilic materials prior to the particle production from the solution. The stabilizing and/or emulsifying agents can also aid in inhibiting and/or preventing phase separation to occur within the solutions of the present invention. Non-limiting examples of suitable stabilizing and/or emulsifying agents include surfactants. The surfactants are able to form a common boundary between the hydrophilic material and the lipophilic material. The surfactants contain polar groups and non-polar groups. In one example, surfactants include those selected from the group consisting of anionic surfactants, nonionic surfactants, amphoteric surfactants, non-lathering surfactants, emulsifiers and mixtures thereof. Non-limiting examples of surfactants useful in the compositions of the present invention are disclosed in U.S. Pat. No. 6,280,757. In addition, there are several emulsifier mixtures that are useful in the present invention. Examples include PROLIPID 141 (glyceryl stearate, behenyl alcohol, palmitic acid, stearic acid, lecithin, lauryl alcohol, myristyl alcohol and cetyl alcohol) and 151 (Glyceryl stearate, cetearyl alcohol, stearic acid, 1-propanamium, 3-amino-N-(2-(hydroxyethyl)-N—N-Dimethyl,N—C(16-18) Acyl Derivatives, Chlorides) from ISP; POLAWAX NF (Emulsifying wax NF), and INCROQUAT BEHENYL TMS (behentrimonium sulfate and cetearyl alcohol) from Croda; and EMULLIUM DELTA (cetyl alcohol, glyceryl stearate, peg-75 stearate, ceteth-20 and steareth-20) from Gattefosse.

In another example of the present invention, the stabilizing and/or emulsifying agent may be selected from the group consisting of: dialkylquaternary compounds, ester oils, silicone oils, waxes, liquid fatty alcohols and fatty acids, microfine particles, and mixtures thereof. One non-limiting ester emulsifier example is glyceryl monooleate.

Non-limiting examples of suitable dialkylquaternary compounds include dialkyl dimethyl quaternaries (e.g. dialkyl(C₁₂-C₁₈) dimethyl ammonium chloride, ditallow dimethyl ammonium chloride, distearyl dimethyl ammonium methyl sulfate) and imidazolinium quaternaries (e.g. methyl-1-oleyl amido ethyl-2-oleyl imidazolinium-methyl sulfate), and mixtures thereof.

Non-limiting examples of suitable ester oils include fatty acid mono and polyesters such as cetyl octanoate, octyl isonanoanate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate; sucrose ester and polyesters, sorbitol ester, and mixtures thereof.

In another example of the present invention, the stabilizing and/or emulsifying agent includes triglycerides, modified triglycerides, synthetic triglycerides, and mixtures thereof. Non-limiting examples of suitable triglycerides include vegetable oils such as jojoba, soybean, canola, sunflower, safflower, rice bran, avocado, almond, olive, sesame, persic, castor, coconut, and mink oils. Non-limiting example of suitable modified triglycerides include ethoxylated and maleated triglyceride derivatives provided they are liquids at 23° C.±2.2° C. Synthetic triglycerides can also be employed provided they are liquid at 23° C.±2.2° C. Proprietary ester blends such as those sold by Finetex as Finsolv are also suitable, as is ethylhexanoic acid glyceride.

Another suitable ester oil is liquid polyester formed from the reaction of a dicarboxylic acid and a diol. Examples of such a liquid polyester include the polyesters marketed by ExxonMobil under the trade name PURESYN ESTER®.

Non-limiting examples of suitable silicone oils and waxes include polydimethyl siloxane, organo functional silicones (alkyl and alkyl aryl, copolyol), and amino silicones.

Non-limiting example of suitable liquid fatty alcohols include those having from about 10 to about 30 carbon atoms. These liquid fatty alcohols may be straight or branched chain alcohols and may be saturated or unsaturated alcohols. Liquid fatty alcohols are those fatty alcohols which are liquid at 25° C. Non-limiting examples of these compounds include oleyl alcohol, palmitoleic alcohol, isostearyl alcohol, isocetyl alcohol, and mixtures thereof.

Non-limiting examples of suitable fatty acids include those having from about 10 to about 30 carbon atoms. These fatty acids can be straight or branched chain acids and can be saturated or unsaturated. Suitable fatty acids include, for example, oleic acid, linoleic acid, isostearic acid, linolenic acid, ethyl linolenic acid, arachidonic acid, ricinolic acid, and mixtures thereof.

Non-limiting examples of suitable microfine particles as surface actives include microfine particles that are dispersible both in water and in oil. The average diameter of the particles used is from about 1 nm to about 200 nm. Advantageous particles are all those which are suitable for stabilizing water-in-oil Pickering emulsions. The amphiphilic characteristics can also be achieved with the surface treatments of these microfine particles. Non-limiting examples of microfine particles include metal oxides and boron nitrides. Non-limiting surface coatings include silicones, silicone derivatives, aluminium hydroxide, and alumina.

### Highly Compressible Fluid

The term “highly compressible fluid” as used herein is defined by way of the reduced temperature (T_{reduced}) and the reduced pressure (P_{reduced}) of the fluid (in pure form) used as a highly compressible fluid. With

$${T}_{reduced} = \frac{T [ K ]}{{T}_{critical} [ K ]}$$

$$and$$

$${P}_{reduced} = \frac{p [ bar ]}{{p}_{critical} [ bar ]}$$

a fluid is defined in the present application as being highly compressible if its reduced temperature is in a range of 0.5 to 2.0 and/or in the range of 0.8 to 1.7 and its reduced pressure is between 0.3 and 8.0. The highly compressible fluid may thus be subcritical with regard to temperature and supercritical with regard to pressure or vice versa or may be subcritical with regard to both temperature and pressure or may be supercritical with regard to both temperature and pressure, or it may be at the critical point.

Suitable highly compressible fluids are a whole series of substances. Non-limiting examples of suitable highly compressible fluids include carbon dioxide, short-chain alkanes, dinitrogen monoxide, nitrogen and mixtures thereof. However, in principle, it is possible to use the vapor phase of any of the substances mentioned in Table 1, and mixtures of these substances, as highly compressible fluid.

TABLE 1

| | Boiling | Critical | Critical | Critical |
|---|---|---|---|---|
| | Point | Temperature | Pressure | Density |
| Compound | (° C.) | (° C.) | (bar) | (kg/m³) |
| CO₂ | −78.5 | 31.3 | 72.9 | 0.448 |
| NH₃ | −33.35 | 132.4 | 112.5 | 0.235 |
| H₂O | 100.00 | 374.15 | 218.3 | 0.315 |
| N₂O | −88.56 | 36.5 | 71.7 | 0.45 |
| CH₄ | −164.00 | −82.1 | 45.8 | 0.2 |
| Ethane | −88.63 | 32.28 | 48.1 | 0.203 |
| Ethylene | −103.7 | 9.21 | 49.7 | 0.218 |
| Propane | −42.1 | 96.67 | 41.9 | 0.217 |
| Propylene | −47.4 | 91.9 | 45.4 | — |
| n-Butane | −0.5 | 152.0 | 37.5 | — |
| i-Butane | −11.7 | 134.7 | 35.9 | — |
| n-Pentane | 36.1 | 196.6 | 33.3 | 0.232 |
| Benzene | 80.1 | 288.9 | 48.3 | 0.302 |
| Methanol | 64.7 | 240.5 | 78.9 | 0.272 |
| Ethanol | 78.5 | 243.0 | 63.0 | 0.276 |
| Isopropanol | 82.5 | 235.3 | 47.0 | 0.273 |
| Isobutanol | 108.0 | 275.0 | 42.4 | 0.272 |
| Chlorotrifluoro- | −31.2 | 28.0 | 38.7 | 0.579 |
| methane | | | | |
| Monofluoromethane | 78.4 | 44.6 | 58.0 | 0.3 |
| Toluene | 110.6 | 320.0 | 40.6 | 0.292 |
| Pyridine | 115.5 | 347.0 | 55.6 | 0.312 |
| Cyclohexane | 80.74 | 280.0 | 40.2 | 0.273 |
| Cyclohexanol | 155.65 | 391.0 | 25.8 | 0.254 |
| o-Xylene | 144.4 | 357.0 | 35.0 | 0.284 |

One or more of the materials within the solution into which the highly compressible fluid is dissolved may initially be a solid rather than a liquid. If it is a solid, then the solid is transformed into a liquid as a result of the highly compressible fluid dissolving within the solution under pressure of at least 50 bars. The mass ratio between the highly compressible fluid and the solution into which the highly compressible fluid is dissolved may be from about 0.1:1 to about 4:1.

In order to fully understand the present invention it is necessary to appreciate what is meant by dissolving or solubilizing a highly compressible fluid in a liquid or a solid substance.

### Non-Limiting Example

A shell material (e.g. petrolatum), which is solid at room temperature, is molten and stored in a storage vessel above its melting temperature. A liquid glycerin is stored in a different storage vessel, at about 23° C.±2.2° C. Both liquids are pumped by high pressure dosing pumps to a static mixer. Heated carbon dioxide is added to the shell material and glycerin solution and subsequently everything is mixed and blended in the static mixer. The carbon dioxide is at least partly dissolved in the solution under high pressure conditions (50-300 bars). Afterwards the mixture is expanded through a single path spray nozzle into a spray tower, which is operated at ambient pressure. Fine droplets are formed in the spray, as carbon dioxide at ambient pressure is no longer appreciably soluble in the still liquid shell material. Thus, during expansion carbon dioxide bubbles are formed and are “leaving” the droplets by breakup. At the same time, carbon dioxide is rapidly cooled down to low temperatures due to the pressure drop (Joule-Thomson phenomenon). The fine droplets are thereby cooled and the shell material solidifies and covers the micro-droplets of the core material (glycerin). Pulverous composites are generated and are collected at the bottom of the spray tower. The gaseous carbon dioxide is cleaned by a cyclone and is exhausted. FIG. 6 shows a simplified flow scheme of that process.

The state and grip of the obtained product at room temperature depends on the properties of the educts. When the used shell material is liquid or sticky at room temperature, the product is highly probable also. When a “dry,” non-sticky powder is of interest, a shell material with a clearly defined melting point is preferable. It is also possible to produce liquid droplets with glycerin enclosed, but these liquid, sticky droplets may have to be collected in a solvent in the spray tower, prior to collision and coalescence. For the encapsulation of glycerin in petrolatum a difference in surface tension is preferable, as the substance with a higher surface tension will be the core material. It is likely, that glycerin exhibits a higher surface tension than the chosen petrolatum, which is in favor for a successful encapsulation of glycerin in petrolatum.

### Test Methods

Unless otherwise indicated, all tests described herein including those described under the Definitions section and the following test methods are conducted on samples that have been conditioned in a conditioned room at a temperature of 23° C.±2.2° C. and a relative humidity of 50%±10% for 2 hours prior to the test. Further, all tests are conducted in such conditioned room.

### Particle Size Test Method

The average particle size of a particle is measured using a Horiba LA-910 commercially available from Horiba International Corporation of Irvine, Calif.

One skilled in the art knows that the suitable and appropriate operating conditions for the Horiba LA-910 can be found by running one or more pilot runs on the Horiba LA-910 for the particle sample. Visually, one skilled in the art can determine whether the particle sample is bimodal or unimodal regarding particle size. If the particle sample contains agglomerates, then one of skill in the art will utilize ultrasonics to break up the agglomerates before measuring the particle size. During the pilot run(s), whether the particle sample is bimodal or unimodal can be determined. During the pilot runs, one skilled in the art can determine the appropriate agitation and circulation speed, and if the average particle size from the particle sample is less than 10 μm, can obtain the relative refractive index from Horiba's database.

Follow the Horiba LA-910 Instrument manual for setup and software use instructions. Obtain the relative refractive index for the particle sample to be tested from the Horiba refractive index database.

Input the appropriate measurement conditions into the instrument: Agitation and Circulation Speed—obtained from pilot run(s); Sampling Times 25; Standard Distribution; Dispersant Tank B; Dispersant Volume 200 ml; Dispersant Volume per Step 10 ml; Dilution Point 10%; Rinse Circulation Time 10 seconds; Rinse Repeat Times 1; Rinsing Volume 100 ml; Relative Refractive Index; Good Range Lower Limit 88%; and Good Range Upper Limit 92%.

Drain the cell of the instrument and add 150 mL of the dispersant to the cell and circulate, sonicate for 2 minutes and agitate. If the cell looks clean and the background reading looks flat, run a blank by pressing “Blank.” Add the solid additive sample to be tested to the cell while the dispersant is agitating and circulating. Continue to add the solid additive sample slowly until the % Transmission of the laser is 90±2 (around 1 mL). Allow the particle sample to circulate through the cell for 2 minutes. After the particle sample has circulated for 2 minutes, press “Measure” to analyze the particle sample. Once the particle sample is analyzed, print the graph and table. Press “Drain” to drain the cell. Rinse the system three times with deionized water using agitation and sonication for 30 seconds each time. For subsequent particle samples, repeat steps 2-10. The laser alignment (four triangles) should be checked between particle samples. The results are reported as follows: 1) a standard resolution histogram for a unimodal distribution or a sharp resolution histogram for a multi-modal distribution; and 2) the Average Particle Size (Mean Diameter).

### Contact Angle Test Method

The contact angle of a material is measured using a DAT 1100 FIBRO system commercially available from Thwing-Albert Instrument Company of West Berlin, N.J.

The syringe and tubing of the DAT 1100 FIBRO system are rinsed with Millipore 18 M′Ω Water 3 times. The syringe is then loaded with Millipore 18 M′Ω Water and any air bubbles are eliminated from the syringe before inserting into the DAT 1100 FIBRO system. The DAT 1100 FIBRO system is calibrated with the calibration standard provided by the manufacturer. The materials are handled with clean tweezers and cotton gloved hands to ensure minimum contact with the measured surface of the material. For each material tested, a total of at least 10 contact angle measurements are taken. The contact angle is reported as the average contact angle measured at 5 s for a material.

The following conditions are used for the DAT 1100 Fibro system: 1) Liquid is Millipore 18 M′Ω Water; 2) Timeout is 0.2 minutes; 3) Number of Drops is 2-3 (per strip); 4) Drop size is 4 microliter; 5) Stroke pulse is 11; 6) Time collected is 0.10 s, 5 s and 10 s; 7) Steps is 1; 8) Minimum height is 8; 9) Minimum width is 10; 10) Capture Offset is 0; 11) Travel time is 2; 12) Pump delay is 5; 13) References Lines; 14) Mod threshold is 0; 15) Cannula Tip is 245; 16) Drop bottom is 97; and 17) Paper Position is 8, 18) Application Mode 1.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A non-ingestible particle comprising at least two incompatible materials produced by a PGSS process.

2. The particle according to claim 1 wherein the average particle size is less than 500 μm as measured according to the Particle Size Test Method described herein.

3. The particle according to claim 1 wherein the average particle size is greater than 1 nm as measured according to the Particle Size Test Method described herein.

4. The particle according to claim 1 wherein the particle exhibits a Morphology Coefficient F of greater than 0.2.

5. The particle according to claim 1 wherein at least one of the incompatible materials comprises a hydrophilic agent.

6. The particle according to claim 5 wherein the hydrophilic agent is selected from the group consisting of: water, humectants, electrolytes, sugar amines, vitamins, natural extracts, peptides, aldehydes, ketones, hexamidines, dehydroxy acetic acids, dihydroxy acetone, water-soluble polymers, water-swellable polymers, colorants, derivatives thereof, salts thereof, and mixtures thereof.

7. The particle according to claim 6 wherein the hydrophilic agent comprises a humectant.

8. The particle according to claim 7 wherein the humectant comprises glycerin.

9. The particle according to claim 1 wherein the hydrophilic agent is at least partially encapsulated by the lipophilic agent.

10. The particle according to claim 9 wherein the lipophilic agent is selected from the group consisting of: ester lipids, hydrocarbon lipids, silicone lipids, fatty alcohols, fatty acids, and mixtures thereof.

11. The particle according to claim 1 wherein the lipophilic agent is at least partially encapsulated by the hydrophilic agent.

12. The particle according to claim 11 wherein the hydrophilic agent comprises polyethylene glycol.

13. The particle according to claim 1 wherein the particle further comprises a coating material.

14. The particle according to claim 1 wherein the particle further comprises a carrier to which the particle is attached.

15. A non-ingestible particle comprising a hydrophilic agent and a lipophilic agent.

16. The particle according to claim 15 wherein the average particle size is less than 500 μm as measured according to the Particle Size Test Method described herein.

17. The particle according to claim 15 wherein the average particle size is greater than 1 nm as measured according to the Particle Size Test Method described herein.

18. The particle according to claim 15 wherein the particle exhibits a Morphology Coefficient F of greater than 0.2.

19. The particle according to claim 15 wherein the hydrophilic agent is selected from the group consisting of: water, humectants, electrolytes, sugar amines, vitamins, natural extracts, peptides, aldehydes, ketones, hexamidines, dehydroxy acetic acids, dihydroxy acetone, water-soluble polymers, water-swellable polymers, colorants, derivatives thereof, salts thereof, and mixtures thereof.

20. The particle according to claim 19 wherein the hydrophilic agent comprises a humectant.

21. The particle according to claim 20 wherein the humectant comprises glycerin.

22. The particle according to claim 15 wherein the lipophilic agent comprises petrolatum.

23. The particle according to claim 15 wherein the particle further comprises a coating material.

24. The particle according to claim 15 wherein the particle further comprises a carrier to which the particle is attached.

25. A plurality of non-ingestible particles comprising at least two incompatible materials produced by a PGSS process.

26. The plurality of particles according to claim 25 wherein greater than 80% of the plurality of particles exhibit a particle size of between 200 μm and 500 nm as measured according to the Particle Size Test Method described herein.

27. The plurality of particles according to claim 25 wherein the plurality of particles exhibit an average particle size distribution from about 250 μm to 100 nm as measured according to the Particle Size Test Method described herein.

28. A plurality of non-ingestible particles comprising a hydrophilic agent and a lipophilic agent produced by a PGSS process.

29. A process for making a non-ingestible particle, the process comprising the step of depressurizing a solution comprising at least two incompatible materials and a highly compressible fluid dissolved in the solution such that a particle comprising the at least two incompatible materials is produced.

30. The process according to claim 29 wherein the solution is produced by dissolving a highly compressible fluid in a solution comprising the at least two incompatible materials.

31. The process according to claim 30 wherein the solution into which the highly compressible fluid is dissolved is produced by mixing a first material with a second material that is incompatible with the first material.

32. The process according to claim 31 wherein the first material comprises a hydrophilic agent.

33. The process according to claim 32 wherein the second material comprises a lipophilic agent.

34. The process according to claim 29 wherein the process further comprises the step of coating the particle with a coating material.

35. The process according to claim 29 wherein the process further comprises the step of mixing a carrier with the particle.

36. A consumer product comprising a particle according to claim 1.

37. The consumer product according to claim 36 wherein the consumer product is selected from the group consisting of: shampoos, body washes, laundry detergents, dishwashing detergents, anhydrous liquid products, bar soaps, paper products, cosmetics, lotions, skin treating products, and mixtures thereof.
